(19) United States
(12) Patent Application Publication
(10) Pub. No.: US 2024/0379210 A1
(43) Pub. Date: Nov. 14, 2024

(54) A massage coaching method, device and application thereof

(71) Applicant: Nutricia Early Life Nutrition (Shanghai) Co., Ltd., Shanghai (CN)

(72) Inventors: Mengjin Liu, Shanghai (CN); Bingzhi Guo, Shanghai (CN); Yi Jin, Shanghai (CN); Jiangnan Wang, Shanghai (CN); Nan Sun, Shanghai (CN)

(73) Assignee: Nutricia Early Life Nutrition (Shanghai) Co., Ltd., Shanghai (CN)

(21) Appl. No.: 18/557,302

(22) PCT Filed: May 4, 2021
(86) PCT No.: PCT/CN2021/091815

§ 371 Date: Oct. 26, 2023

(51) Int. Cl. G16H 20/30; A61H 7/00; G09B 5/06; G09B 19/00

(52) U.S. Cl. CPC G16H 20/30; A61H 7/00; G09B 5/065; G09B 19/003

(57) ABSTRACT

The present invention related to a method for massage coaching which comprises: capturing at least one image; displaying the at least one image; identifying a person in the at least one image; identifying at least one target massage position of the person in the at least one image; displaying at least one massage indication at the at least one target massage position.

### FIELD OF THE INVENTION

The present invention relates to a massage coaching method, device and application thereof. The massage may be a self-breast-massage or any other types of massage.

### BACKGROUND

Many moms intend to breast feed longer to give the best immunity to their babies. However, only a small number of moms continues breastfeeding in the first six months. During breastfeeding, many moms don't have sufficient breastmilk, while many of them think breast feeding is painful. Proper breast massage during the pregnant period and lactating period after giving birth can help to overcome or at least ease these issues. Proper and regular breast massage may improve the health of women as well, for example, preventing breast cancer, etc.

However, not all moms have the time, energy and resource to hire private coaches to teach them the correct way to massage the breasts for such issues. Thus, an easy and convenient self-breast-massage coaching method, device and application are needed. The same problem applies to other types of massages as well, e.g., on a hand, face, leg, foot, etc. Especially for easy/simple relaxing/physiotherapeutic massages that can be performed by oneself, which are needed by a user, e.g., a woman, a man, an old person, etc. Additional examples of massages and related physical therapies specifically including, but not limited to grip-strength and acupressure, may include muscle development and mobility enhancing (e.g., healthy aging).

The present invention helps a user to learn correct massage methods at home thereby improving their health, e.g., self-breast-massage to improve and/or breastfeeding experience.

### SUMMARY OF THE INVENTION

The present invention relates to a massage coaching method, device and application thereof. In this application breast massage is given as an example, however, other types of massage is covered by the present invention as well, e.g., massage for on a hand, face, leg, foot, etc. For example, the breast massage may be a self-massage, e.g., a self-breast-massage.

According to an embodiment of the present invention, a method for massage coaching comprises capturing at least one image; displaying the at least one image; identifying a person in the at least one image; identifying at least one target massage position of the person in the at least one image; displaying at least one massage indication at the at least one target massage position.

According to an embodiment of the present invention, an electronic device comprises a camera, a display, and a processer to perform the method of the present invention.

According to an embodiment of the present invention, a storage medium and a program/software are configured to store instructions executed by at least one processor to perform the method of the present invention.

### BRIEF DESCRIPTION OF THE DRAWINGS

The present invention will be discussed in more detail below, with reference to the attached drawings, in which:

FIG. 1 shows a method for massage coaching according to the present invention.

FIG. 2A shows a user interface according to the present invention.

FIG. 2B shows a user interface according to the present invention.

FIG. 3 shows a device for massage coaching according to the present invention.

### DESCRIPTION OF EMBODIMENTS

Embodiments of the present disclosure will be described herein below with reference to the accompanying drawings. However, the embodiments of the present disclosure are not limited to the specific embodiments and should be construed as including all modifications, changes, equivalent devices and methods, and/or alternative embodiments of the present disclosure.

The terms “have,” “may have,” “include,” and “may include” as used herein indicate the presence of corresponding features (for example, elements such as numerical values, functions, operations, or parts), and do not preclude the presence of additional features.

The terms “A or B,” “at least one of A or/and B,” or “one or more of A or/and B” as used herein include all possible combinations of items enumerated with them. For example, “A or B,” “at least one of A and B,” or “at least one of A or B” means (1) including at least one A, (2) including at least one B, or (3) including both at least one A and at least one B.

The terms such as “first” and “second” as used herein may modify various elements regardless of an order and/or importance of the corresponding elements, and do not limit the corresponding elements. These terms may be used for the purpose of distinguishing one element from another element. For example, a first printing form and a second printing form may indicate different printing forms regardless of the order or importance. For example, a first element may be referred to as a second element without departing from the scope the present invention, and similarly, a second element may be referred to as a first element.

It will be understood that, when an element (for example, a first element) is “(operatively or communicatively) coupled with/to” or “connected to” another element (for example, a second element), the element may be directly coupled with/to another element, and there may be an intervening element (for example, a third element) between the element and another element. To the contrary, it will be understood that, when an element (for example, a first element) is “directly coupled with/to” or “directly connected to” another element (for example, a second element), there is no intervening element (for example, a third element) between the element and another element.

The expression “configured to (or set to)” as used herein may be used interchangeably with “suitable for,” “having the capacity to,” “designed to,” “adapted to,” “made to,” or “capable of” according to a context. The term “configured to (set to)” does not necessarily mean “specifically designed to” in a hardware level. Instead, the expression “apparatus configured to . . . ” may mean that the apparatus is “capable of . . . ” along with other devices or parts in a certain context.

The terms used in describing the various embodiments of the present disclosure are for the purpose of describing particular embodiments and are not intended to limit the present disclosure. As used herein, the singular forms are intended to include the plural forms as well, unless the context clearly indicates otherwise. All of the terms used herein including technical or scientific terms have the same meanings as those generally understood by an ordinary skilled person in the related art unless they are defined otherwise. The terms defined in a generally used dictionary should be interpreted as having the same or similar meanings as the contextual meanings of the relevant technology and should not be interpreted as having ideal or exaggerated meanings unless they are clearly defined herein. According to circumstances, even the terms defined in this disclosure should not be interpreted as excluding the embodiments of the present disclosure.

According to the present invention, the massage may be a self-massage, in which the person who performs the massage is the person who receives the massage. For example, the person may massage her breast by her own hands according to the present invention, or perform other type of massages, e.g., on a hand, face, leg, foot, etc.

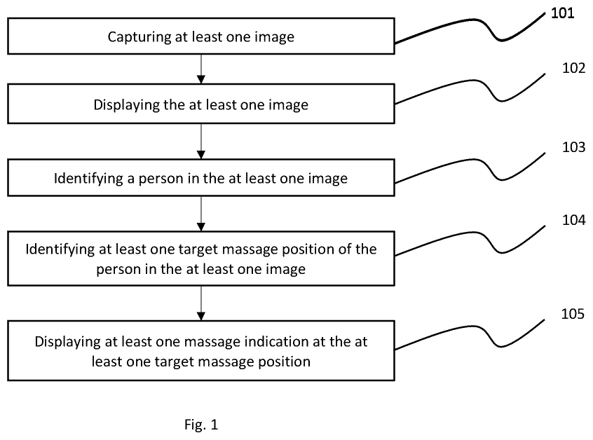

The massage in the present invention may also be performed by another person (e.g., according to the coaching method). For example, a masseuse, a husband or a nurse may perform the massage to a patient according to the present invention. E.g., a nurse may follow the coaching method in FIG. 1 and may massage the breast of a female who is pregnant or lactating. The present invention may be applied to men as well. For example, a man may follow the coaching method according to the present invention, e.g., a breast massage to prevent breast cancer, recover from breast cancer, or for any other health and non-health related purposes, a leg massage to relax after physical excises, or a physiotherapeutic hand massage after a hand injury. Additional examples of massages and related physical therapies specifically including, but not limited to grip-strength and acupressure, may include muscle development and mobility enhancing (e.g., healthy aging).

FIG. 1 shows a method for massage coaching. Some steps shown in FIG. 1 may be omitted and the order of the steps may be in a different sequence. In the below explanation a breast massage is used as an example, however, other types of massage are included in the scope of the present invention as well.

In step 101, at least one image (used interchangeably with “the image”, “the captured image”, “the captured images” and “the images” in this document) is captured. This may be via a separate camera, or an integrated camera in an electronic device, e.g., a mobile phone, a tablet, a laptop, a desktop, a TV, etc. The image may be captured continuously or one image in each predetermined period (e.g., one image in each second).

Before this step, a user (e.g., in this application, the user may be a female, or someone else assisting the female) may start the massage coaching application installed (and/or stored) in the electronic device (for example, a breast massage app on a smart phone). Then the device may identify (e.g., performed by at least one processor) whether there is a usable camera. If no usable camera is identified, the method stops, or displays at least one (breast) massage guiding video/guiding photo/images, which may have been pre-filmed and each may show a correct massage manner. Each of the (breast) massage guiding videos (or photos/images) may be according to a different massage type. The guiding videos may include both moving images and audio information, which may be pre-recorded. Guiding photos or images may be used to replace the guiding videos, e.g., drawings, cartoon image, or a pre-taken photo. The at least one guiding videos is used interchangeable with the guiding video, the guiding videos, the video and the videos in this application. The same interchangeable using applies to the guiding photos/images.

The massage type may be input by the user via an I/O unit of the device, or determined according to other information, e.g., at least one predetermined condition. For example, if the massage is a breast massage, the predetermined condition may be at least one of one or a plurality of pregnancy stages and/or one or a plurality after birth stages, such as an early pregnant stage (0 to 14 weeks), a mid-term pregnant stage (14 Weeks to 28 weeks), a late pregnant stage (28 Week to before giving birth), feeding stage (0 to 4 days after giving birth), and etc.

A massage type may include a massage manner on a target massage positon and/or the massage function. The massage manner may include how to apply a massage, e.g., a massaging direction, strength, etc. The target massage position may be a body part where the massage is applied. The massage function may be the effect of the massage, e.g., relaxing, therapeutic, etc.

If it is breast massage type, the massage type may further include types of at least one of a circling massage, a side pushing massage, a direct pushing massage, a relaxing massage, a lactating promoting massage, a residual milk reduction massage, and a breast shaping massage. For example, a circling massage may be to massage a breast or around the breast in a circling manner. A side pushing massage may be to press and slide forward on one or two sides of the breast. A direct pushing massage may be to press and slide forward in all directions of the breast. Other massage types are obvious from their names (i.e., providing the indicated function) and would be understood by a skilled person. As another example, a foot massage type may further include relaxing finger massage, therapeutic palm massage, etc.

If a usable camera is detected, the user may be asked to agree on using the camera. For example, after the user inputs to agree on using the camera, later steps are performed. Otherwise, the same steps as no camera identified are performed.

Before and/or during steps 101 and/or step 102, the user may be notified with some general guidance, e.g., “please keep a certain distance to the camera/device (e.g., 0.5 to 3 meters, or 1 meter)”; “the method will count down five seconds before capturing images”, “please relax and sit/lying comfortably”, etc.

In step 102, the image is displayed on a screen/display of the device, for example on a separate display/monitor/screen, or an integrated display unit, e.g., on a mobile phone, a tablet, a laptop and so on. A single captured image or a video formed by the captured images may be displayed, e.g., a live stream captured by the camera.

The image may be displayed as mirrored which helps the user to find the massage position (e.g., on/around the breast) easily according to the indication discussed later in this document.

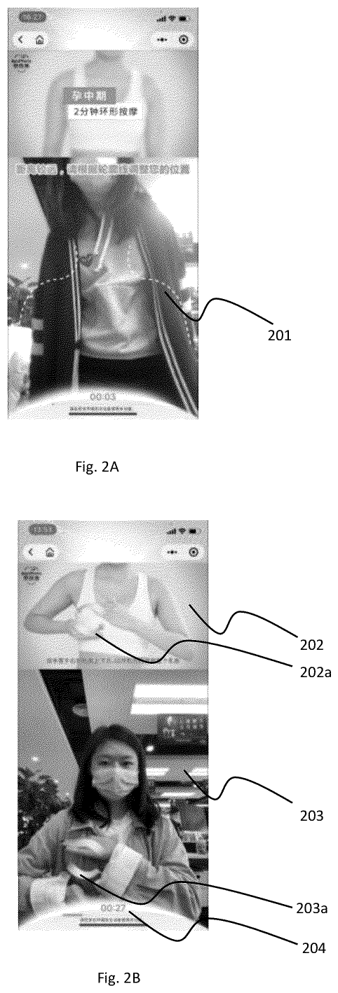

In Step 102, the at least one (e.g., breast) massage guiding video (referred also as the guiding video(s) and the video(s)) may be displayed in a predetermined area of a screen/display, which overlaps or is separate from a predetermined displaying area for the at least one captured/displayed image. For example, as in FIGS. 2A and 2B, the guiding video may be displayed in an upper area of the screen 202 and the at least one image is displayed in a lower area 203. The video and the image may be resized to fit to the predetermined areas. The videos may start playing in this step or ready to be started. The video may start playing after receiving an input of the user, and/or, in step 103 after a person is identified in the image (and/or is identified in ready to massage position). If the guiding videos is playing after a person is identified (e.g., during the massaging process), if the person left (e.g., cannot determined anymore) during the video, the guiding videos may be paused. The guiding videos may be resumed when the person is identified again.

In the present application, guiding photos or images may be used to replace the guiding videos, e.g., drawings, cartoon image, or a pre-taken photo, wherever guiding videos are discussed in this application.

A use may also input directly and/or adjust the display areas of the video and the image, and may also zoom in and/or zoom out the video and the image within the predetermined areas in any step of the present invention.

The guiding videos/photos/images may have been pre-filmed and each may show a correct massage manner. Each of the guiding videos may be according to a different massage type as discussed for step 101.

The at least one guiding video/photo/film may be displayed in any or all steps of the present invention.

Step 103 may identify whether a person (e.g., the one who receives the massage) appearing in the image and/or whether the person being in a proper posture for massage. The step of identifying of whether a person appears in the image may be omitted, and the step of identifying of whether the person is in a general massage posture may be omitted as well. If no person appears in the image (e.g., no body part of a person is identified for longer than a predefined time period), the device may notify the user to adjust the camera or the position of the user in order to be in the image, e.g., via at least one of an audio, image, a movie or any other notification method, and/or the device may start playing the guiding videos/photos/images. The guiding videos may be paused if a person is identified in a later period and the normal steps are performed (e.g., steps 103, 104, 105 and their alternatives). If the guiding videos is playing after a person is identified (e.g,. during the massaging process), if the person left (e.g., cannot determined anymore) during the video, the guiding videos may be paused. The guiding videos may be resumed when the person is identified again.

If it is identified that a person appears (e.g., there is one or more of parts of a person are identified) in the image, the device may further identify whether the person is in a general massage posture. For example, a guiding outline as shown in FIG. 2A may be displayed to guide the user to pose correctly, which may prevent the person being too far from, too close to or not in the middle of the image.

The identifying of the general massage posture being met, may include identifying that at least one or more basic rules (or referred as the rule, the rules, filtering rules, or basis filtering rules) is met by the posture of the user. A guiding video may start playing after receiving an input of the user, and/or, after a person is identified in the image (and/or is identified in ready to massage position). If the guiding videos is playing after a person is identified (e.g., during the massaging process), if the person left (e.g., cannot determined anymore) during the video, the guiding videos may be paused. The guiding videos may be resumed when the person is identified again.

A rule may be considered to be met, if the related posture is according to the rule for longer than or equal to a predefined period, or longer than or equal to a threshold period in a predefined period. For example, a rule may be considered to be met if the posture is according to it for 5 seconds, or the rule may be considered to be met if the posture is according to the rule for longer than or equal to 3 seconds in a total period of 5 seconds.

The basic rules may include:

1. The person is within certain area of the image, e.g., shoulders are in the lower half of the image, the upper body of the person appears in the lower half of the image, and/or the upper body of the person is within the guiding outline area.

2. The person is not too close to and facing the camera. For example, the upper body of the person is within the guiding outline area and cover more than at least certain percentage of the area. For another example, if the user is not showing a side of the body then she is not facing the camera directly. This may be determined according to the shoulder width of the person and distance of the ears, and/or the length of arms in the image. For example, if the shoulder width in the image is shorter than 1.2 times of the distance of the person, the device determines that the person is too close to the camera. The mom may be notified to turn to the camera and keep a longer distance.

3. The camera is not positioned too low or too high. For example, the device may determine according to the shoulder width and the hip width of the mom in the image. For instance, the camera is too high if the shoulder width is more than 1.2 times of the hip width, and/or the camera is too low if the hip width is more than 1.5 times of the shoulder width. The mom may be notified to higher or lower the camera accordingly.

The body parts of the person mentioned above may be identified/recognized and/or abstracted as key points and/or key areas. A key point may be a position on a central point of the concerned body part, or on a side point of the concerned body part, or a random position within the concerned body part area. For example, key points may include at least one of a right hand, right wrist, right elbow, right shoulder, nose, left palm, left wrist, left elbow, left shoulder, left breast, right breast, left nipple, right nipple, etc. The recognizing/identifying of the key points of a body may be via artificial intelligence and/or image reorganization technologies. A key area may be an area of a concerned body part, e.g., a back area, a head area, a right hand area, right wrist area, right elbow area, right shoulder area, nose area, left palm area, left wrist area, left elbow area, left shoulder area, left breast area, right breast area, left nipple area, right nipple area, etc.

The key point of a hand may be the center point of the back of the hand; the key point of a wrist may be the middle point of a line between the hand and forearm; the key point of an elbow may be the joining point of the forearm and upper arm; the key point of a shoulder may be the joining point of the upper arm and the shoulder; the key point of a head may be the nose (e.g., for the mom) or the ear drop (e.g., for the baby); the key point of a breast may be the nipple position, and/or may be estimated according to the width of the shoulder and upper body length of the mom; the key point of hips may be the middle point or one of the two edge points of the hip bones; the key point of a neck may be the joining point of the upper body and head.

This step may include a step of identifying the posture of the person via key points/key area on the user in the image.

The guiding outline may be displayed during and throughout step 103 and/or step 104. The identified key points/areas may also be displayed or not displayed. When displaying the key points/areas, the key points may be connected in a structure of a human being and/or the key area may be displayed with a colored area or a box/circle/outline of the key area. The displayed guiding outline and/or key points/areas can help the user to understand the relative positions of the body parts, correct her posture and/or find the massage position easier. The outline and/or key points/areas may be displayed (e.g., mirrored) at the beginning period of step 103, the whole step, or until the end of the method.

During Step 103, at least one (e.g., breast) massage guiding video/photo/image may be displayed as in other steps. A guiding video may start playing after receiving an input of the user, and/or, after a person is identified in the image (and/or is identified in ready to massage position). If the guiding videos is playing after a person is identified (e.g., during the massaging process), if the person left (e.g., cannot determined anymore) during the video, the guiding videos may be paused. The guiding videos may be resumed when the person is identified again.

The basic rules may include key point/area information on a correct massage posture of the user. The plurality of rules may include different sets of rules based for key point/area information in different massage types. For example, if it is a breast massage, a side pushing massage and a direct pushing massage may have different or the same rules. The correct massage posture may be obtained via artificial intelligence after training with a large numbers of images with correct massage postures, for example, by subtracting all the key points/areas and obtaining relative position information (and/or in acceptable ranges) of the key points/areas.

In Step 104, at least one target massage position may be identified in the at least one image, which may be the position to be massaged (e.g., according to the coaching/guiding videos/photos/images and/or a massaging type). The target massage position may be associated with a body part of the person receiving the massage, e.g., a hand position, a finger position, a breast positon of the person, both breast positions of the person, a position around a breast of the person, or any other positions that may need to be massaged.

The target massage position may be according to the massage type and/or guiding videos. For example, the target massage position may be a specific position around the breast areas according to a specific massage type.

The target massage position may change from a first positon to a second position during one massage period, and the position change may be according to the coaching/guiding videos and/or a massaging type. For example, from massaging left breast to the right breast, or from massaging the top of a breast to the area around the breast.

The target massage position may be identified according to image analysis, e.g., a direct recognition of a body part(s) that needs to be massaged. For example, a breast may be recognized for massaging.

The target massage positon may be identified according to relations with other body parts, e.g., in the at least one image. For example, a breast position may be determined/identified according to at least one key points of the shoulders and/or chin of the person. For example, if an identified shoulder key point is the central part of the corresponding shoulder, e.g., the middle point between the closer edge of the neck and the outer edge of the shoulder. Then, the target massage position for the breast may be identified as a certain distance vertically below the key point of the shoulder, for example, certain pixels (e.g., 200 pixels or 500 pixels), or certain absolute distance on or percentage of the length of the screen (e.g., 1.5 cm, 2 cm or 2.5 cm, or 10% to 30% of the length of the screen). The length of the screen may be the length of the screen parallel to the line between the shoulder key point and the target massage position. For example, if the screen is set up in a portrait direction (e.g., the longer edge is vertical), the length of the screen is the length of the longer edge of the screen; if the screen is set up in a landscape direction (e.g., the shorter edge is vertical), the length of the screen is the length of the shorter edge of the screen.

In Step 105, at least one massage indication may be displayed on the at least one target massage position that has been identified. For example, the indication may be an image, text, an animation, or in any other forms that can indicate a massage manner.

The indication may indicate at least one of a massage direction, a massage position, a massage strength and a massage timing. For example, indicating a massage direction by an arrow, a hand or a moving animation, indicating a massage strength by different colours (e.g., red is strong, green is light) or text, and indicating the massage timing with text or a clock shaped figure/animation. The massage timing may be the massage time left or past. An example of the direction indication (with two arrows) is shown in 203a of FIG. 2B, and an example of the timing indication is shown in 204 of FIG. 2B.

The at least one guiding video/photo/image may be displayed (e.g., start playing) simultaneously in step 105, and at least one further massage indication may be simultaneously displayed on the video according to a correct massaging manner, e.g., at least one of the direction, strength, position and timing. The further indication(s) may be incorporated in the videos when filing the videos, or may be added afterwards on top of the videos when displaying the videos. A user may start the massage according to the indication and the guiding video/photo/image, e.g., as shown in FIG. 2B. A guiding video may start playing after the indication is display and/or the further indication is displayed. If the guiding videos is playing after a person is identified (e.g., during the massaging process), if the person left (e.g., cannot determined anymore) during the video, the guiding videos may be paused. The guiding videos may be resumed when the person is identified again.

The indication and the further indication may be in the same form but in different positions on the screen. The indication may be at the target massage position of the captured image of the person, but the further indication may be at the correct massage position on the guiding video/photo/image. The indication and the further indication may be in different forms as well, e.g., in different colors to distinguish from each other.

The steps in 101 to 105 may be performed iteratively until the method ends according to at least one condition. The iteration may be once in a predefined time period, e.g., once in every second.

The method may end when a predefined time period is identified, e.g., the predefined massage time period or the total time period of the guiding video, expires. The method may end by a user input, e.g., closing the application, turn off the screen, power off the device, etc. The method may end if no person can be identified in the image anymore for longer than a predetermined time period. The method may end if all the massage videos have been played, e.g., all massages have been performed.

A series of massages may be performed before it taking effect. According to the present invention, notifications may be output to remind the user to perform a massage. For example, the present invention may use the calendar on a smart phone to remind the user, or a timer in the application may be used to trigger the notification. The user may also set the notification system via the user interface by herself.

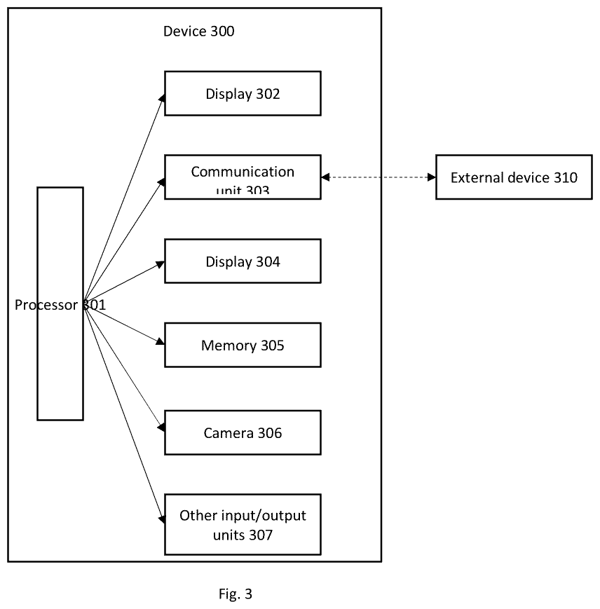

FIG. 3 shows a device 300, e.g., a mobile phone, tablet, laptop, desktop, smart watch, a TV, etc., to perform the present invention.

The device 300 may comprise a processor 301, a display 302, a communication unit 303, a memory 305, a camera 306 and other input/output units 307.

The processor 301 is configured to perform the program/instructions stored in the memory 305, e.g., via controlling other components such as the display 302, the communication unit 303, the memory 305, the camera 306 and other input/output units 307.

The displayed 302 may be controlled by the processor 301 to perform the all the displaying function (and input function if it is a touch screen) in the present invention such as in steps 102 and 105.

The communication unit 303 may be controlled by the processor 301 to perform all communication function in the present invention. For example, if an external device 310 (e.g., an sever) is used to perform some of or all the identifying functions in the present invention, e.g., steps 103 and 104, messages are communicated via the communication unit 303, such as transmitting the images and/or receiving the identifying results. Optionally, the image may be not stored in the external device 310 for privacy reasons, e.g., after each identifying step, the image is immediately deleted in the external device 310.

The memory 305 is configured to store the massage coaching application software and/or data. For example, the basic rules, the captured images, identified results, user profile, usage history, massaging records, guiding vides, massaging schedule and other necessary information, etc. The application may provide at least one entry for the user to check/overviewing these data.

The camera 306 is configured to capture images in the present invention, e.g., in steps 101.

The other input/output units 307 are configured to perform other input/output functions of the present invention, for example, to output audio notifications when playing the guiding videos, or receive user input for the massage type and/or any other user settings.

In the present invention, at least a part of the device (e.g., FIG. 3) or method (e.g., FIG. 1) or user interface (e.g., FIG. 2) may be implemented as instructions stored in a non-transitory computer-readable storage medium, e.g., in the form of a program module, a piece of software, a mobile app, and/or other forms. The instructions, when executed by a processor (e.g., the processor 301), may enable the processor to carry out a corresponding function according to the present invention. The non-transitory computer-readable storage medium may be the memory 305.

According to an embodiment of the present invention, a method for massage coaching comprises: capturing at least one image; displaying the at least one image; identifying a person in the at least one image; identifying at least one target massage position of the person in the at least one image; displaying at least one massage indication at the at least one target massage position.

According to an embodiment of the present invention, the at least one image may be mirrored when displayed, and/or the at least one target massage position may be associate with a position of a body part.

According to an embodiment of the present invention, the method may be performed iteratively once in a predefined time period.

According to an embodiment of the present invention, the method, may further comprise: receiving an input to select a massage type, and/or determining a massage type according to at least one predetermined condition, herein the at least one massage indication and/or the at least one target massage position is according to the massage type.

According to an embodiment of the present invention, the massage type may be according to at least one of one or a plurality of pregnancy stages and/or one or a plurality of after birth stages.

According to an embodiment of the present invention, the massage type may include at least one of a circling massage, a side pushing massage, a direct pushing massage, a relaxing massage, a lactating promoting massage, a residual milk reduction massage, and a breast shaping massage.

According to an embodiment of the present invention, the displaying of the at least one image may comprise displaying a guiding outline for guiding a pose of the person.

According to an embodiment of the present invention, the identifying of the person may include identifying whether the person poses according to the guiding outline.

According to an embodiment of the present invention, the at least one target massage position may be identified according to at least one shoulder position of the person.

According to an embodiment of the present invention, the method further may comprise displaying a massage guiding video simultaneously.

According to an embodiment of the present invention, at least one further massage indication may be displayed in the guiding video.

According to an embodiment of the present invention, the least one massage indication and/or the at least one further massage indication may indicate at least one of a massage direction, a massage position, a massage strength and a massage timing.

According to an embodiment of the present invention, the method may further comprise ending the massage when a predefined time period expired and/or receiving an input to end the massage.

According to an embodiment of the present invention, an electronic device comprising a camera, a display, and a processer to perform the method according to the method of the present invention.

According to an embodiment of the present invention, a storage medium configured to store instructions executed by at least one processor to perform the method of the present invention.

1. A method for massage coaching comprising:
capturing at least one image;
displaying the at least one image;
identifying a person in the at least one image;
identifying at least one target massage position of the person in the at least one image;
displaying at least one massage indication at the at least one target massage position.

2. The method in claim 1, wherein the at least one image is mirrored when displayed, and/or wherein the at least one target massage position is associated with a position of a body part.

3. The method in claim 1, wherein the method is performed iteratively once in a predefined time period.

4. The method in claim 1, further comprising:
receiving an input to select a massage type, and/or
determining a massage type according to at least one predetermined condition,
wherein the at least one massage indication and/or the at least one target massage position is according to the massage type.

5. The method in claim 4, wherein the massage type is according to at least one of one or a plurality of pregnancy stages and/or one or a plurality of after birth stages.

6. The method in claim 5, wherein the massage type includes at least one of a circling massage, a side pushing massage, a direct pushing massage, a relaxing massage, a lactating promoting massage, a residual milk reduction massage, and a breast shaping massage.

7. The method in claim 1, wherein the displaying of the at least one image comprises displaying a guiding outline for guiding a pose of the person.

8. The method in claim 7, wherein the identifying of the person includes identifying whether the person poses according to the guiding outline.

9. The method in claim 1, wherein the at least one target massage position is identified according to at least one shoulder position of the person.

10. The method in 1, further comprising displaying a massage guiding video simultaneously.

11. The method in claim 10, wherein at least one further massage indication is displayed in the guiding video.

12. The method in claim 1, wherein the least one massage indication and/or the at least one further massage indication indicate at least one of a massage direction, a massage position, a massage strength and a massage timing.

13. The method in claim 1, further comprising ending the massage when a predefined time period expired and/or receiving an input to end the massage.

14. An electronic device comprising
a camera configured to capture at least one image;
a display configured to:
display the at least one image, and
displaying at least one massage indication at the at least one target massage position; and
a processer configured to:
identify a person in the at least one image;
identify at least one target massage position of the person in the at least one image.

15. (canceled)

16. The electronic device in claim 14, wherein the at least one image is mirrored when displayed, and/or wherein the at least one target massage position is associated with a position of a body part.

17. The electronic device in claim 14, wherein the method is performed iteratively once in a predefined time period.

18. The electronic device in claim 14, further comprising:
an input unit configured to receive an input to select a massage type, and/or
wherein the processor is further configured to determine a massage type according to at least one predetermined condition, wherein the at least one massage indication and/or the at least one target massage position is according to the massage type.

19. The electronic device in claim 18, wherein the massage type is according to at least one of one or a plurality of pregnancy stages and/or one or a plurality of after birth stages.

20. The electronic device in claim 19, wherein the massage type includes at least one of a circling massage, a side pushing massage, a direct pushing massage, a relaxing massage, a lactating promoting massage, a residual milk reduction massage, and a breast shaping massage.

21. The electronic device in claim 14, wherein the display of the at least one image comprises is further configured to display a guiding outline for guiding a pose of the person.
